(19) United States
(12) Patent Application Publication
(10) Pub. No.: US 2015/0164928 A1
(43) Pub. Date: Jun. 18, 2015

(54) PHARMACEUTICAL COMPOSITION FOR THE REGENERATION OF THE LIVER

(71) Applicant: Phrontier SARL, Caudebec-en Caux (FR)

(72) Inventor: Martin Goerne, Hamburg (DE)

(73) Assignee: Phrontier SARL, Caudebec-en Caux (FR)

(21) Appl. No.: 14/407,784

(22) PCT Filed: Jun. 14, 2013
(86) PCT No.: PCT/EP2013/001781

§ 371 Date: Dec. 12, 2014

(30) Foreign Application Priority Data
Jun. 15, 2012 (EP) 12004527.3

(51) Int. Cl. A61K 31/675; A61K 31/519

(52) U.S. Cl. CPC A61K 31/675; A61K 31/519

(57) ABSTRACT

A pharmaceutical composition for use in a method for liver regeneration or for enhancement of the liver performance comprises the vitamins B6, B9, and B12 in the daily doses: B6 not less than 4 mg, optionally not more than 500 mg; B9 not less than 0.6 mg, optionally not more than 15 mg; B12 not less than 0.008 mg, optionally not more than 4 mg.

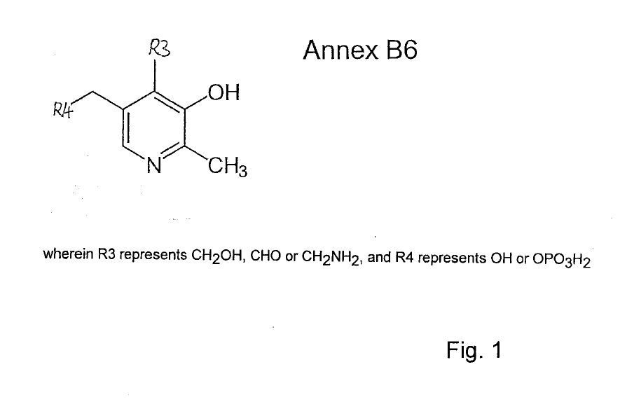

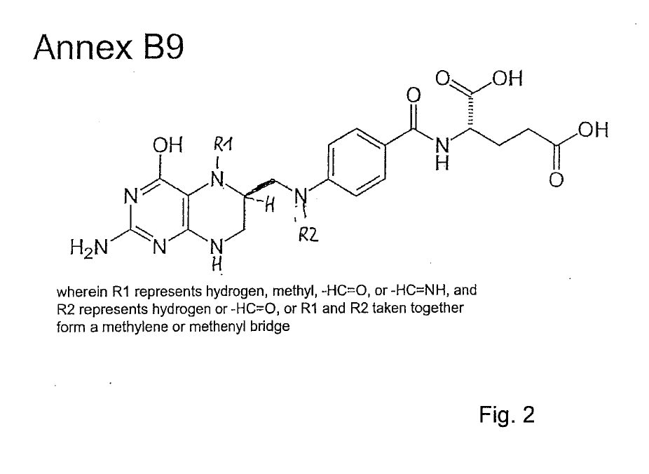

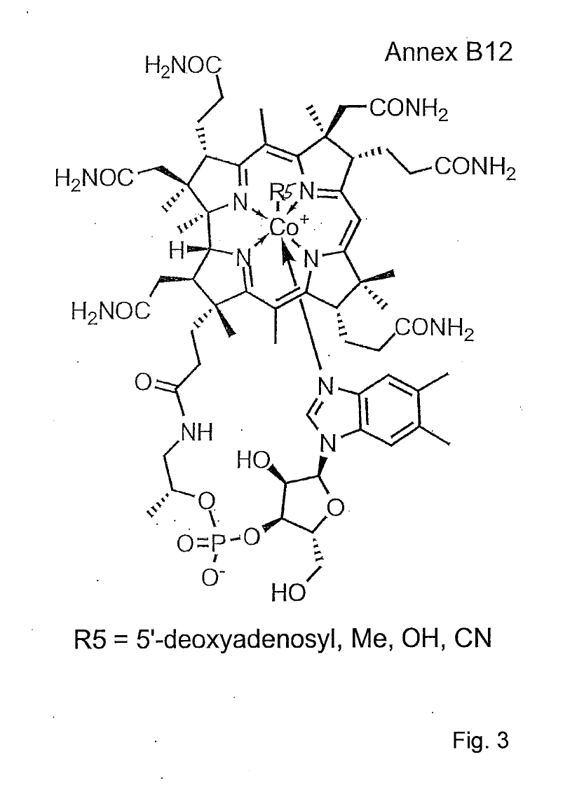

The present invention relates to a composition for the enhancement of the performance of the human liver, in particular a cirrhotic liver.

Certain compositions are already known, to which a healing effect on damaged livers of mammals, in particular of man, has been ascribed, e. g. from documents EP 0 101 294 A2 or WO 03/066058 A1. None of the compositions proposed in these documents has actually fulfilled the hopes pinned thereon. There is therefore still a demand for a composition capable of permanently alleviating a clinically manifest functional disorder of the human liver, e. g. caused by a noxa. In man, ethanol is often the causal noxa; nevertheless, ethanol abuse is widespread in Europe (and not only there).

It is therefore an aim of the invention to provide a pharmaceutical composition for the regeneration of the liver or for enhancement of the performance of the liver. This object is solved by the composition for therapeutic use according to claim 1.

The invention starts out from the observation that certain vitamin combination drugs effected a decrease of elevated liver enzyme levels in serum. Extended studies on patients with damaged livers and, associated therewith, elevated blood levels of the pertinent liver enzymes led to the finding, on which the present invention is based, that in particular a relatively accurately defined range of the relative amounts of the three components B6, B 12 and folic acid of the vitamin mixture, and a likewise accurately defined range of the total daily dose, has a marked effect on the liver enzyme serum levels. In a narrower range within this liver-therapeutically clinically effective range, a beneficial effect on the liver function was determined even when the daily dose was significantly reduced, namely down to as much as one sixth.

It was furthermore determined that within this narrower range, a synergistic effect was present with the dosing of such plant extracts containing e. g. polyphenolic antioxidants in sizeable amounts. The preparation of such extracts is described in European patent document EP 1 190 024 B1. The polyphenols include at least 2, in embodiments 5 to 7 phenolic rings, and in total at least 5, in embodiments at least 12 phenolic hydroxyl groups.

The liver performance is in this context understood as, on the one hand, a detoxificating performance, measurable as the reduction of an elevated level of bilirubin, and on the other hand, as a synthetic performance, measurable as the increase of a previously depressed albumin level in the serum. Both are causally connected.

In the following, both absolute weight values for the daily dose of the vitamin components are used, as also relative ones obtained by referring to RDA (Recommended Daily Allowance) values. The RDA values used herein are 1.3 mg for vitamin B6, 2.4 μg for vitamin B12, and 0.4 mg for vitamin B9 (folic acid). In particular, daily doses for the vitamins B6, B9 (folic acid) and B12 are proposed which range from 1.5 times to 40 times the RDA (B9), from 3 times to 380 times (B6) or even from 3 times to 1700 times (B12), respectively. Herein, in embodiments a weight ratio of the vitamins B9 (folic acid) and B12 is in the range 0.8:1 or more, in particular the range 6:1 to 10:1. The weight ratio of the vitamins B6 and B12 is in the range 40:1-1000:1, in particular 55:1-80:1. The daily dose for the vitamins B6, B9 (folic acid) and B12 are generally not below 4 mg (B6), not less than 0.6 mg (B9) and not less than 0,008 mg (B12), respectively. It is furthermore desirable to administer not more than 500 mg (B6), or not more than 15 mg (B9) not more than 4 mg (B12) daily of the respective vitamins. The daily doses refer to an adult of average weight.

In some embodiments, in which the weight proportion of the vitamin B6 is reduced to less than 95% or less than 90%, and the weight proportion of the vitamin B9 is increased to more than 5% or more than 9% (wherein the total weight proportion of the vitamin B12 is always marginal, namely is between 0.1% and 2%), a daily dose of less than 60 mg/d of the composition down to 8 mg/d is sufficient with regard to a reduction of the total serum bilirubin and, associated therewith, an improvement of the subjectively determinable well-being. The efficacy can, in this dosage range, be significantly enhanced further through the additional administration of at least 25 mg/d of a polyphenolic antioxidant, e. g. in form of grape seed extract or aronia extract. without vitamin administration, such dietary supplements are without any measurable effect on the liver function.

In the context of this application, “vitamin B6” shall be understood as one or more from the group pyridoxin, pyridoxal, pyridoxamin, as well as their esters, in particular phosphoric acid esters (see Annex B6). “Vitamin B9 (folic acid)” shall be understood as one or more from the group folic acid, folate, and their metabolites, namely the derivates listed in Annex B9 as well as their Esters and Amides. “Vitamin B12” shall be understood as one or more from the group of Cobalamines, as listed in Annex B12. The group members each represent pharmaceutically acceptable salts or derivates of the basic compound.

### 1. EXAMPLE

According to a first example, a composition with vitamin B6, folic acid and vitamin B12 in the ratio 50:1:1 (each in mg) over a duration of 8 weeks was administered to a group of 28 patients with elevated liver function levels and elevated total bilirubin level (Child-Index A to B): The bilirubin level decreased on average to 77% of the initial value, GOT to 50%, GPT to 57% and γGT to 78%. After discontinuing the therapy, the initial values were again reached within several weeks.

### 2. EXAMPLE

Then, a composition with vitamin B6, folic acid and vitamin B12 in the ratio 50:6.5:0.75 (each in mg) was administered over a duration of 8 weeks to a subgroup of 10 of these patients: The bilirubin level decreased on average to 63% of the initial value, GOT to 60%, GPT to 58% and γGT to 76%. After discontinuing the therapy, the initial values were again reached within several weeks.

### 3. EXAMPLE

A composition with vitamin B6, folic acid and vitamin B12 in the ratio 100:1:1 (each in mg) was administered over a duration of 8 weeks to a subgroup of 7 of the patients: The bilirubin level decreased on average to 92% of the initial value, GOT to 88%, GPT to 74% and γGT to 89%. After discontinuing the therapy, the initial values were again reached within several weeks.

### 4. EXAMPLE

A composition with vitamin B6, folic acid and vitamin B12 in the ratio 8.0:1.0:0.12 (each in mg) was administered over a duration of 8 weeks to 27 of the patients: The bilirubin level decreased on average to 84% of the initial value, GOT to 68%, GPT to 80% and γGT to 79%. After discontinuing the therapy, the initial values were again reached within several weeks.

### 5. EXAMPLE

The same composition with vitamin B6, folic acid and vitamin B12 in the ratio 50:1:1 (each in mg) as in the 1. example was administered over a duration of 8 weeks to a subgroup of 16 of the patients, but additionally a daily dose of 210 mg of a grape seed extracts with 20% oligocyclic polyphenols: The bilirubin level decreased on average to 68% of the initial value, GOT to 46.5%, GPT to 48% and γGT to 63%. After discontinuing the therapy, the initial values were again reached within several weeks.

### 6. EXAMPLE

The same composition with vitamin B6, folic acid and vitamin B12 in the ratio 8.0:1.0:0.12 (each in mg) as in the 4. example was administered over a duration of 8 weeks to a subgroup of 11 of the patients, additionally a daily dose of 210 mg of the grape seed extract with 20% oligocyclic polyphenols: The total bilirubin level decreased on average to 73% of the initial value, GOT to 59%, GPT to 65% and γGT to 66%. After discontinuing the therapy, the initial values were again reached within several weeks.

### COMPARATIVE EXAMPLES

To a control group of 10 patients, only the grape seed extract was administered, in the same daily dose as described above: Within the eight-week duration of the therapy, no change of the transaminase levels or of the total bilirubin levels was detected. No further change as according to examples 5. and 6. was detected, when the daily dose of an oligocyclic polyphenol was below 25 mg.

### RESULTS

According to the 2. and 3. example, such compositions are effective with respect to a temporary improvement of the liver function, in which the components of the vitamin-B9-group are about as highly concentrated (by weight) as the components of the vitamin-B12-group, and the components of the vitamin-B6-group are about 40- to 120times higher. In particular, such compositions are effective in which the ratio B9/B12 is in the range between 0.8 and 1.2.

According to the 1. and 4. example, compositions are effective with respect to a temporary improvement of the liver function in a much lower concentration, in which the components of the vitamin-B9-group are about 6-10times as concentrated (by weight) as the components of the vitamin-B12-group, and the components of the vitamin-B6-group are about 40- to 60-times higher than those of the B9-group. In this range, the combination of these vitamins is thus synergistic.

According to the 5. and 6. example, compositions are more effective with respect to a temporary improvement of the liver function in the same concentration as according to the 4. example, when additionally at least 25 mg per day of an oligocyclic polyphenol was administered as an antioxidant. In this range, the combination of the vitamins with antioxidants was thus synergistic, too.

The said compositions can be used for liver regeneration, or for enhancement of the liver performance, or for administration to persons with impeded liver function, but without hyper-homocysteinemia, for improving on their well-being. To this end, the daily dose of so-administered vitamin B12 is in the range 0.1-1.2 mg/d; the daily dose of administered vitamin B9 (folic acid) is in the range 1-8 mg/d; and the total daily dose of administered vitamin composition is in the range 8-60 mg/d or 8-20 mg/d (synergistic range).

After discontinuing, the measured serum levels return to their initial values within a few (ca. 5-8) weeks, thus supporting the conclusion that the administration of the described compositions does not result in structural changes in the liver in the sense of healing, but actively influences the metabolic processes in a beneficial manner such that compensation mechanisms are supported. For the same reason, it is paramount to avoid liver toxic noxa such as ethanol for the therapy to be successful.

The invention is not limited to the examples described above, but its scope is determined by the appended claims.

1. A method of regenerating liver or enhancing the liver performance, comprising administering orally to a person in need thereof, a pharmaceutical composition comprising vitamins B6, B9 and B12 in the following daily doses:
B6 not less than 4 mg;
B9 not less than 0.6 mg; and
B12 not less than 0.008 mg.

2. The method according to claim 1, wherein the daily dose of vitamin B9 is in the range 0.6-15 mg/d (1.5-40 RDA) or 0.8-8 mg/d (2-20 RDA).

3. The method according to claim 1, wherein the daily dose of vitamin B12 is in the range 0.008-4 mg (3-1700 RDA) or 0.1-1.2 mg/d (40-500 RDA) or 0.2-1.2 mg/d (80-500 RDA).

4. The method according to claim 1, wherein the daily dose of vitamin B6 is in the range 4-500 mg (3-380 RDA) or 6.5-117 mg/d (5-90 RDA).

5. The method according to claim 1, wherein the pharmaceutical composition comprises the vitamins B6 and B9 in a weight ratio of from 2:1 to 12:1.

6. The method according to claim 5, wherein the pharmaceutical composition comprises the vitamins B9 (folic acid) and B12 in a weight ratio of from 6:1 to 10:1.

7. The method according to claim 1, wherein the pharmaceutical composition comprises the vitamins B6 and B12 in a weight ratio of from 55:1 to 80:1.

8. The method according to claim 7, wherein the pharmaceutical composition comprises the vitamins B9 and B12 in a weight ratio of 7:1-9:1.

9. The method according to claim 8, wherein the weight ratio B6:B9 is in the range 6:1-9:1.

10. The method according to claim 8, wherein the pharmaceutical composition is administered in a total daily dose in the range 8-60 mg/d or 8-20 mg/d.

11. The method according to claim 5, further comprising administering to the person a daily dose of at least 25 mg/d polyphenolic antioxidant.

12. The method according to claim 11, wherein grape seed extract or aronia extract is the source of the polyphenolic antioxidant.

13. The method according to claim 7, wherein the daily dose of vitamin B12 is in the range 0.01-0.6 mg/d (4-250 RDA).

14. The method according to claim 1, wherein the pharmaceutical composition comprises the vitamins B9 and B12 in the weight ratio 4:5 to 5:4 and the daily dose of Vitamin B6 is in the range 40-120 mg/d (30-90 RDA).

15. The method according to claim 1, wherein the pharmaceutical composition is administered to a person with impeded liver function, but without hyper-homocysteinemia, to improve the person's well-being.

16. The method according to claim 1, wherein the pharmaceutical composition comprises the vitamins B6, B9 and B12 in at least one of the following daily doses:
B6 not more than 500 mg;
B9 not more than 15 mg; and
B12 not more than 4 mg.

17. The method according to claim 5, wherein the pharmaceutical composition comprises the vitamins B6 and B9 in a weight ratio of more than 6:1.

18. The method according to claim 5, wherein the pharmaceutical composition comprises the vitamins B6 and B9 in a weight ratio of less than 9:1.

19. A method of treating impeded liver function, comprising administering orally to a person in need thereof, a pharmaceutical composition comprising vitamins B6, B9 and B12 in the following daily doses:
B6 not less than 4 mg;
B9 not less than 0.6 mg; and
B12 not less than 0.008 mg.

20. The method of claim 19, wherein the pharmaceutical composition is administered to a person with impeded liver function, but without hyper-homocysteinemia, to improve the person's well-being.
